(19) United States
(12) Patent Application Publication
(10) Pub. No.: US 2004/0241170 A1
(43) Pub. Date: Dec. 2, 2004

(54) Isolation of cells from neural cell populations using antibodies to fa1/dlk1

(76) Inventors: Charlotte Harken Jensen, Svendborg (DK); Borge Teisner, Odense (DK); Mette Gronborg, Ballerup (DK); Lars U Wahlberg, Ballerup (DK)

Correspondence Address: BROWDY AND NEIMARK, P.L.L.C., 624 NINTH STREET, NW, SUITE 300, WASHINGTON, DC, 20001-5303, US

(21) Appl. No.: 10/487,442

(22) PCT Filed: Aug. 26, 2002
(86) PCT No.: PCT/DK02/00559

(30) Foreign Application Priority Data
Aug. 24, 2001 (US) 60314794

(51) Int. Cl. A61K 39/395

(52) U.S. Cl. 424/178.1

(57) ABSTRACT

The present invention relates to the use of antibodies recognising Fetal Antigen-1 (FA1/dlk1) for the detection and isolation of cell subpopulations from neural cell populations, in particular from cell populations from the central nervous system. In one embodiment, the dopaminergic neurons in the Substantia nigra pars compacta are detected and separated from other cell populations in this region of the brain. In another embodiment, neural stem and progenitor cells are isolated from other more committed cells in the CNS. The isolated cells may be used for transplantation, drug screening, production of cell type specific antibodies, and gene discovery.

### FIELD OF INVENTION

The present invention relates to the use of FA1 antibodies for recognising and isolating subsets of cells originating from fetal and adult mammalian neural cells, including cells of the CNS, which includes neural stem and progenitor cells and their differentiated progeny.

### BACKGROUND OF THE INVENTION

Dopaminergic neurons are located in many regions in the CNS and are characterized by the expression of tyrosine hydroxylase (TH). TH catalyzes the rate-limiting step in the biosynthesis of dopamine utilizing tyrosine, molecular oxygen and tetrahydrobiopterin as co substrates in the formation of 3,4-dihydroxyphenylalanine (DOPA). Dopaminergic neurons derived from the ventral midbrain are of special interest because of the selective loss of this cell population in patients with Parkinson's disease. However, the study of this population of cells has been difficult because of the heterogeneity of cell cultures established from the midbrain. In most instances, dopaminergic neurons comprise five percent or less of the total cell population, which also complicates drug screening and gene discovery based on such cultures. TH expressing neurons may be induced in cultures containing neural stem and progenitor cells (described in pending patent application No. U.S. Ser. No. 60/286,084) or immortalised cell lines established from these. However, also in these cultures only a minor fraction of the cells is induced into the TH expressing phenotype.

Dopaminergic neurons can be visualized in formalin-fixed cell preparations, by immunostaining for tyrosine hydroxylase (TH). However, presently a method to quantify and isolate this specific cell population without harming the cells remains to be established. The method of detection and isolation may be based on the use of antibodies recognising surface antigen(s) present on particular cell populations of interest including the dopaminergic neurons of the midbrain.

The establishment of a method to purify dopaminergic neurons may also be beneficial in the context of implanting developing dopaminergic neurons originating from aborted human fetuses in the brains of patients with Parkinson's disease (Bjorklund, Novartis Found Symp 2000; 231:7-15). Although a successful restoration of function in the patients was observed in many cases, undesirable side-effects were observed in a recent study (Freed et al., 2001 N. Engl. J. Med. 344, 710-9). The problems in this study may be caused by the use of heterogeneous cell populations and uncertainties in number of dopaminergic cells transplanted (Dunnett; Nat Rev Neurosci 2001 May; 2 (5): 365-9).

Fetal antigen 1 (FA1) is one of the increasing numbers of proteins belonging to the epidermal growth factor (EGF)-superfamily that have been identified within the last decade. The protein contains 6 EGF-like repeats and displays a very similar primary structure and level of glycosylation in man, mouse and rat (Jensen C H, et al. Hum Reprod 1993 8(4), 635-641; Jensen C H et al. Eur J Biochem 1994 225(1), 83-92. Bachmann E, et al J Reprod Fertil 1996 107(2), 279-285. Krogh T N, et al. Eur J Biochem 1997 244(2), 334-342. Carlsson H E, et al Biol Reprod 2000 63(1), 30-33.)

FA1 is synthesized as a larger transmembrane precursor and released from cells after proteolytic action of an unidentified enzyme. Several groups have described cDNA clones for this precursor, each assigning a new name for the cDNA depending on the species and tissue/cell type from which they isolated it. As a result, the FA1 precursor has been referred to as adrenal specific mRNA (human pG2 Helman L J. Nucleic Acids Res 1990 18(3), 685), delta-like (mouse and human dlk1 Laborda J, et al. J Biol Chem 1993 268(6), 3817-3820.), preadipocyte factor-1 (mouse, rat and bovine pref-1 Laborda J, et al J Biol Chem 1993 268(6), 3817-3820. Smas C M, Cell 1993 73(4), 725-734; Carlsson C, et al. Endocrinology 1997 138(9), 3940; Fahrenkrug S C, Biochem Biophys Res Commun 1999 264(3), 662-667) and zona glomerulosa-specific factor (rat ZOG Okamoto, et al. Steroids 1997 62(1), 73-76). The official name for the gene encoding this membrane-associated protein is now delta-homologue 1, dlk1 (Gubina, et al. Cytogenet Cell Genet 1999 84(3-4), 206-207.), referring to the close resemblance between its EGF-repeats and those of the transmembrane protein Delta, which was originally described in Drosophila Melanogaster. Delta is one of the ligands for the Notch receptor and interactions between these membrane proteins are crucial for the development of various tissues [Artavanis-Tsakonas, Science 1995 268(5208), 225-232.]. The primary structure of dlk1 does not allow conclusions as to whether it is a ligand or receptor, but both the membrane-associated and the soluble form (i.e. FA1) of the DLK1 gene have been shown to be involved in the differentiation/proliferation processes of various cell types and act through autocrine/paracrine and juxtacrine intercellular signaling (reviewed by Laborda in Laborda J. Histol Histopathol 2000 15(1), 119-129.), the membrane-associated form possibly as a homodimer (Kaneta, J Immunol 2000 164(1), 256-264). Apart from being present in preadipocytes and stromal cells, the expression of FA1/dlk1 in adults seems to be associated with endocrine structures. FA1 has been localized in B-cells of the pancreatic Langerhans' islets (Jensen, Hum Reprod 1993 8(4), 635-641); Jensen, Eur J Biochem 1994 225(1), 83-92; Tornehave, Histochem Cell Biol 1996 106(6), 535], the adrenal gland (medulla and cortex) [Jensen, Hum Reprod 1993 8(4), 635-641], the somatotroph cells of the adenopituitary gland [Larsen, Lancet 1996 347(8995), 191], the sex hormone-producing Leydig cells of the testis, and theca interna and Hilus cells of the ovary [Jensen, Mol Hum Reprod 1999 5(10), 908]. FA1 has also been demonstrated in tumors [Jensen, Eur J Biochem 1994 225(1), 83-92; Tornehave, Histochem Cell Biol 1996 106(6), 535; Harken Jensen, Tumour Biol 1999 20(5), Jensen, Mol Hum Reprod 1999 5(10), 908] including Small Cell Lung Cancer, pheochromocytomas and neuroblastomas.

Expression of FA1 in the adult CNS has been observed (Harken-Jensen Ph.D. thesis; Odense Univ. 1999) and FA1 expression has been observed in the fetal CNS (Floridon et al., Differentiation 2000 66(1), 49-59) both authors gave preliminary results wherein the cellular location of expression was not determined. The possibility of using FA1 targeting to select specific populations from the CNS is not suggested by either of these publications, nor was it realised by the inventors until these recent findings. Furthermore, there are no published reports of FA1 expression in neural stem- and progenitor cultures derived from the CNS. Kuo et al (WO01/57233) report the discovery of cDNA encoding a novel Pref-1 like protein in a cDNA library from human brain. However the protein encoded shares only 39% identity with Pref-1/FA1.

Antibodies to FA1 have been used previously for cell sorting. Bauer, (Molecular And Cellular Biology, p. 5247-5255 Vol. 18, No. 9) used anti-dlk1 polyclonal antiserum for dlk1 detection and flow cytometry analysis of detached stromal cells and pre-B cells. Garcés (Differentiation 1999 64:103-114) used Pref-1 antibodies for flow cytometry analysis and cell sorting of preadipocytes and their differentiated progeny. Uchida et al. (PNAS 2000; 97, 14720) recently reported the isolation of an enriched population of human CNS stem cells from fresh fetal brain by FACS sorting with a number of antibodies. Cells sorted for CD133⁺, 5E12⁺, CD24⁻, CD34⁻, CD45⁻, were able to initiate neurosphere cultures in-vitro, and differentiate into neurons and glia. Antibody screening revealed that the hematopoietic stem cell marker, CD133, was expressed on 90-95% of neurosphere cells. In contrast the antibodies of the present invention label only a small sub-population of neurosphere cells.

### SUMMARY OF THE INVENTION

The present invention relates to a method of obtaining a cell population enriched or diminished in FA1 (dlk1) expressing cells comprising the steps of:

a) combining a starting population containing cells originating from mammalian neural cells with antibodies which bind specifically to FA1 to produce a first cell mixture,

b) removing unbound antibodies from the first cell mixture to produce a second cell mixture, and

c) separating cells comprising FA1 antibodies from the second cell mixture to produce a cell population enriched in FA1 expressing cells and a cell population diminished in FA1 expressing cells.

The present invention concerns antibodies that recognize the FA1 antigen that is expressed as a membrane-associated protein in specific populations of cells of the mammalian CNS; in cultures containing mammalian neural stem- and progenitor cells; and in culture of in-vitro differentiated stem- and progenitor cells. An example of an antibody used in the invention is the mouse monoclonal FA1 antibody (clone 142.2) or a mono-specific polyclonal anti-FA1 antibody. The FA1 antibody binds to a subset of mature neurons present in the adult human and rodent brain and to sub-populations of cells in cultures containing and/or derived from neural stem- and progenitor cells.

The invention also concerns a method for preparing a cell population useful for cell transplantation that is enriched or diminished in dopaminergic, noradrenergic or serotonergic neurons, or immature cells of the CNS, which population may also be substantially free of other types of neural cells.

The invention also concerns therapeutic materials and methods for transplanting dopaminergic, noradrenergic and serotonergic neurons or immature cells of the CNS that can be used in the treatment of neurodegenerative diseases such as Parkinson's disease, Alzheimer's disease and Huntingdon's chorea; and the effects of Cerebral Ischaemia and Stroke.

The present invention also provides cell populations enriched in dopaminergic neurons, neural progenitor cells or neural stem cells, which are important vehicles for ex-vivo gene therapy.

The cell populations provided by the invention may also be used in encapsulated devices for the treatment of neurodegenerative diseases such as Parkinson's disease and Huntingdon's chorea. They may also be used in drug screening, for the generation of cell-type specific antibodies and in gene discovery.

The present invention is based on the experimental finding that a subset of cells originating from the CNS expresses FA1 and that there exists a strong relationship between FA1 expressing cells in this subset on the one side and cells having one or more of the following characteristics on the other side: neural stem cells, neural progenitor cells, TH positive cells, monoaminergic cells, serotonergic cells and dopaminergic cells. The present invention is further based on the recognition that the feature of FA1 expression may be used as a basis for selecting and isolating cells having one or more of the said characteristics.

### BRIEF DESCRIPTION OF THE FIGURES

FIG. 1 shows FA1 immunostainings of a sectioned sphere containing proliferating human neural stem- and progenitors using the polyclonal rabbit anti-FA1 antibody and DAB protocol. A representative field using a 10× objective is shown in 1A and a selected field using a 100× objective in 1B.

FIG. 2 shows FA1 immunostaining of the proliferating adherent stem cell line HNSC.100 cell line using the polyclonal rabbit anti-FA1 antibody and Cyanin-3 conjugated goat-anti-rabbit IgG. A representative field using a 20× objective is shown.

FIG. 3 shows FA1 antigen expression on the surface of the proliferating culture of the lateral ganglionic eminence using the polyclonal rabbit anti-FA1 antibody and Cyanin-3 conjugated goat-anti-rabbit IgG. A representative field using a 40× objective is shown.

FIG. 4 shows cultures of human neural progenitors established from human fetal forebrain (10wFBr991013) plated onto PLL/laminin coated coverslips in N2 medium containing aFGF (100 ng/ml), forskolin (25 μM), TPA (100 nM) and dbcAMP (100 μM). After 3 days incubation, cells were fixed and immunostained for FA1 (A) or TH (B). A representative field using a 40× objective is shown.

FIG. 5 depicts cultures of human neural progenitors established from human fetal forebrain (10wFBr991013) plated onto PLL/laminin coated coverslips in N2 medium containing aFGF (100 ng/ml), forskolin (25 μM, TPA (100 nM) and dbcAMP (100 μM). After 3 days incubation, cells were fixed and cell-surface labeled for FA1. A representative field using a 20× objective is shown.

FIG. 6 depicts cross-sections through the adult rat midbrain.

FIG. 6A shows staining for FA1. Many FA1-positive cells are seen throughout the substantia nigra pars compacta (SNc) and the ventral tegmental area (VTA). Strongly FA1-immunoreactive cells are also seen in the Edinger-Westphal nucleus (EW).

FIGS. 6D-G depict double labelling for FA1 and tyrosine hydroxylase and show a large number of TH+ as well as FA1⁺ neurons in the substantia nigra pars compacta (D-E). In higher magnification (F-G) of the boxed areas in D and E, co-localisation of TH⁺ and FA1⁺ is observed in the neurons.

FIGS. 6B-C show FA1 staining of the adult rat brain following unilateral injection of 6-hydroxydopamine. FA1 positive terminals in the striatum (terminal area for nigral dopaminergic neurons) are shown to be absent around the site of injection (arrow in FIG. 6C) confirming the identification of these neurons by FA1 antibodies.

FIG. 7 shows cross-sections through the embryonic mouse midbrain at three different stages of development E10.5, 11.5 and 12.5 with TH labeling and with FA1 labeling.

FIG. 8 shows FACSVantage analysis of FA1 expression in proliferating neurospheres derived from the embryonic human forebrain. FIG. 8A shows selection of a subpopulation of FA1-positive cells in dissociated proliferating neurospheres using the FA1 antibody (2.2%=R2). FIG. 8B shows that 0.2% of the negative control cells are located in R2.

FIG. 9 shows FACSVantage analysis of FA1 expression in proliferating neurospheres derived from the fetal human forebrain. FIG. 9A shows selection of a subpopulation of FA1-positive cells in dissociated proliferating neurospheres using the FA1 antibody (0.5%=β2). FIG. 9B shows that 0.1% of the negative control cells are located in R2.

FIG. 10 shows FACSVantage analysis of FA1 expression in TH-induced neurospheres derived from the fetal human forebrain.

FIG. 10A shows selection of a subpopulation of FA1-positive cells in dissociated TH-induced neurospheres using the FA1 antibody (4.4%=R2). FIG. 10B shows that 0.5% of the cells in the negative control are located in R2.

FIG. 11 shows FACSVantage analysis of FA1 expression in the immortalized neural stem cell line HNSC.100. FIG. 11A shows selection of a subpopulation of FA1-positive cells in dissociated proliferating HNSC.100 population using the FA1 antibody (2.6%=R2). FIG. 11B shows that 0.2% of the cells in the negative control are located in R2.

FIG. 12 shows a repetition of FACSVantage analysis of FA1 expression in the immortalized neural stem cell line HNSC.100. FIG. 12A shows selection of a subpopulation of FA1-positive cells in dissociated proliferating HNSC.100 population using the FA1 antibody (0.7%=R2). FIG. 12B shows that 0.1% of the cells in the negative control are located in R2.

FIG. 13 shows FA1 staining of the HNSC.100 cell line after cell sorting for FA1 immunoreactivity using the FACSVantage with the polyclonal rabbit anti-FA1 antibody and Cyanin-3 conjugated goat-anti-rabbit IgG. FIG. 13A depicts FA1 staining of HNSC.100 cells positively sorted for expression of FA1, FIG. 13B depicts FA1 staining of HNSC.100 cells negatively sorted for expression of FA1.

### DETAILED DESCRIPTION OF THE INVENTION

The present invention relates to the use of antibodies that can bind to the FA1 antigen. More specifically it concerns the use an antibody, referred to herein as “FA1 Ab” that facilitates the isolation of specific populations of neural cells derived from the mammalian CNS. These isolated cell populations make possible improved techniques e.g. for transplantation, drug screening and gene discovery. The isolated cells may also be employed to produce panels of monoclonal antibodies to specific populations of cells originating from the neural system. The isolated cell populations of the invention can also be employed in ex vivo gene therapy. The isolated cell populations may be further sorted based on the expression of other lineage specific markers.

Initial studies detected FA1/dlk1 mRNA expression by in situ hybridization in several monoaminergic nuclei of the adult rat and human mesencephalon and pons, more specifically in the ventral tegmental area/substantia nigra pars compacta (VTA/SNc) area and the locus coeruleus (LC). These monoaminergic nuclei contain neurons, which produce the neurotransmitters Dopamine and Norepinephrine. The expression of FA1/dlk1 in the VTA/SNc and LC was verified by immunohistochemistry with FA1 Ab. Furthermore, FA1 immunoreactivity was detected in neurons of the Edinger-Westphal nuclei (EW), in the pons and in the dopaminergic neurons in the reticular part of Substantia Nigra (SNr) in the mesencephalon.

The expression of FA1/dlk1 in the substantia nigra pars compacta (SNc) and ventral tegmental area (VTA) is of particular interest in relation to Parkinson's disease, which is caused by a progressive degeneration and loss of dopaminergic neurons in these nuclei, predominantly SNc. Thus, FA1/dlk1 appears to be a marker protein for dopaminergic neurons. So far, no specific surface marker has been described for this population.

Dopaminergic cells have been previously identified by their expression of the soluble enzyme Tyrosine Hydroxylase (TH) in combination with Aromatic Amino Acid Decarboxylase (AADC). A further differentiation is required since noradrenergic cells also express both TH and AADC. Thus dopaminergic cells are positively identified by their expression of TH and a lack of expression of Dopamine-β-hydroxylase (DBH), which is present in noradrenergic cells. The expression of these enzymes can be visualised by immuno-histochemical staining and/or in-situ hybridisation.

FA1/dlk1 expression was also detected in proliferating cell cultures containing neural stem- and progenitor cells derived from the CNS. Cultures containing neural stem and progenitor cells can be established from a number of regions of the developing and the mature (adult) brain and expanded in vitro as described in by Carpenter (U.S. Pat. No. 6,103,530; incorporated herein by reference). These cultures can be grown as non-adherent clusters (“neurospheres”) under serum-free conditions in the presence of epidermal growth factor (EGF), basic fibroblast growth factor (bFGF) and leukaemia inhibitory factor (LIF). When plated on a substrate like laminin in medium without mitogens, they differentiate and have the ability to generate the major phenotypes in the CNS, neurons, astrocytes and oligodendrocytes.

Furthermore, TH expressing neurons can be induced in the cultures by using a differentiation protocol described in pending patent application No. U.S. Ser. No. 60/286,084. FA1 stainings of TH induced cultures showed that cells displaying strong FA1 immunoreactivity are observed but absent in control cultures that have not been exposed to TH inducing conditions. Staining was localised intracellularly and in the processes. These cells may be sorted using an FA1 antibody allowing an identification and/or enrichment for a specific subpopulation of the differentiated cells, which may include the TH expressing cells. To date there has been no cell surface marker available by which these populations of cells can be sorted.

In connection with the present invention the expression “population of cells originating from mammalian neural cells” means any population of cells, which originates from neural tissue including any population of cells cultured in vivo, differentiated in vivo or derived in any other way.

In a preferred embodiment of the invention the starting cell population is selected from the group consisting of:

(i) a population of primary neural cells,

(ii) a population of in vitro cultured neural stem- and/or progenitor cells, and

(iii) a population of in vitro differentiated neural stem and/or progenitor cells.

In connection with the present invention the expression “neural cells” means cells of any neural tissue in a mammal, including cells from the Central Nervous System (CNS) and the peripheral and autonomic nervous system, including cells of the adrenal medulla and ganglion cells of the gut.

The expression “primary neural cells” means cells as collected from the mammal without any in vitro cultivation thereof. The population of primary neural cells may be any mixture of cells. Preferably, the population of neural cells collected from the mammal is in the form of a cell suspension, wherein the cells have been dissociated so as to be present as single cells. The dissociation may be effected by any conventional method and equipment suitable for dissociation, such as mechanical dissociation.

In a preferred embodiment of the method of the invention, the cells of the starting population originate from human tissue. The cells of the starting population may originate from both fetal and adult tissue, preferably fetal tissue. Preferably, the cells of the starting population originate from the CNS and from subdissected fragments thereof.

The FA1 specific antibodies may be polyclonal or monoclonal antibodies, preferably the FA1 specific antibodies are monoclonal.

Preferably, the FA1 specific antibodies are labelled. In one embodiment of the invention the separation of cells comprising FA1 antibodies is carried out by a mechanical cell sorter.

In a preferred embodiment of the invention the FA1 specific antibodies are coupled to a fluorescent labelling compound. In this case the separation of cells comprising FA1 antibodies is preferably carried out using a fluorescense-activated cell sorter (FACS).

In a further preferred embodiment of the invention the antibodies are biotinylated. A particularly preferred variant of this embodiment is a method, wherein prior to step c) the second cell mixture is contacted with a streptavidin-fluorochrome or a avidin-flurochrome, and wherein the separation of cells comprising FA1 antibodies is carried out using a fluorescense-activated cell sorter (FACS). An alternative variant of the said embodiment is a method, wherein prior to step c) the second cell mixture is contacted with streptavidin or avidin linked to a particle, and wherein the separation of cells comprising FA1 antibodies is carried out by separating the particulate phase from the liquid phase.

In a further preferred embodiment of the invention, the antibodies are linked to a solid particle. Preferably, the solid particle is a magnetic particle. In this embodiment of the invention, the separation of cells comprising FA1 antibodies is preferably carried out by separating the particulate phase from the liquid phase.

A further preferred embodiment of the invention is a method, wherein prior to step c) the second cell mixture is contacted with an antibody to the FA1 specific antibody linked to a particle, and wherein the separation of cells comprising FA1 antibodies is carried out by separating the particulate phase from the liquid phase. Preferably, the particle is a magnetic particle.

A further preferred embodiment of the invention is a method, wherein the cells of the starting population are adherent cells cultivated on a solid support, and wherein the removal of unbound antibodies is carried out by rinsing.

A further preferred embodiment of the invention is a method, wherein the cells of the starting population are cultivated in suspension, and wherein the removal of unbound antibodies is carried out by centrifugating the first cell mixture and separating off the resulting supernatant.

A further preferred embodiment of the invention is a method, wherein the starting cell population is subjected to a further cell sorting procedure to enrich or diminish the cell population in cells expressing at least one further lineage specific marker. The further lineage specific marker may i.a be nestin, glial fibrillary acidic protein (GFAP), vimentin, CD133, β3-tubulin and tyrosine hydroxylase (TH), 5E12⁺, CD24⁻, CD34⁻, CD45⁻. Preferably, the further lineage specific marker is CD133.

The antibodies of the subject invention can be labelled according to standard methods known in the art. For example, antibodies can be labelled with detectable labels such as fluorescein, rhodamine or with radioactive isotopes, or with biotin. Biotin binds strongly and irreversible to avidin. Biotinylated antibodies may be visualized by incubation with conjugates consisting of horseradish perioxidase and biotin bound to avidin followed by detection of the enzymatic activity using a chromogenic substrate.

Alternatively, biotinylated antibodies may be incubated with a streptavidin-flurochrome.

Cell-Sorting Techniques

The ability to recognise dopaminergic cells with antibodies allows not only for the identification and quantification of these cells in tissue samples, but also for their separation and enrichment in suspension. This can be achieved by a number of cell-sorting techniques by which cells are physically separated by reference to a property associated with the cell-antibody complex, or a label attached to the antibody. This label may be a magnetic particle or a fluorescent molecule. The antibodies may be cross-linked such that they form aggregates of multiple cells, which are separable by their density. Alternatively the antibodies may be attached to a stationary matrix, to which the desired cells adhere.

Various methods of separating antibody-bound cells from unbound cells are known. For example, the antibody bound to the cell (or an anti-isotype antibody) can be labelled and then the cells separated by a mechanical cell sorter that detects the presence of the label. Fluorescence-activated cell sorters are well known in the art. In one embodiment, the anti-FA1 antibody is attached to a solid support. Various solid supports are known to those of skill in the art, including, but not limited to, agarose beads, polystyrene beads, hollow fiber membranes, polymers, and plastic petri dishes. Cells that are bound by the antibody can be removed from the cell suspension by simply physically separating the solid support from the cell suspension. Preferred protocols, however, will be described.

Super paramagnetic nanoparticles may be used for cell separations. The microparticles are coated with a monoclonal antibody for a cell-surface antigen. The antibody-tagged, super paramagnetic microparticles are then incubated with a solution containing the cells of interest. The microparticles bind to the surfaces of the desired cells, and these cells can then be collected in a magnetic field.

Selective cytophoresis can be used to produce a cell suspension from mammalian brain containing dopaminergic neurons. The cell suspension is allowed to physically contact, for example, a solid phase-linked monoclonal antibody that recognizes an antigen on the desired cells. The solid-phase linking can comprise, for instance, adsorbing the antibodies to a plastic, nitrocellulose, or other surface. The antibodies can also be adsorbed on to the walls of the large pores (sufficiently large to permit flow-through of cells) of a hollow fiber membrane. Alternatively, the antibodies can be covalently linked to a surface or bead, such as Pharmacia Sepharose 6 MB macrobeads. The exact conditions and duration of incubation for the solid phase-linked antibodies with the CNS cell suspension will depend upon several factors specific to the system employed. The selection of appropriate conditions, however, is well within the skill of the art.

The unbound cells are then eluted or washed away with physiologic buffer after allowing sufficient time for the stem cells to be bound. The unbound cells can be recovered and used for other purposes or discarded after appropriate testing has been done to ensure that the desired separation had been achieved. The bound cells are then separated from the solid phase by any appropriate method, depending mainly upon the nature of the solid phase and the antibody. For example, bound cells can be eluted from a plastic petri dish by vigorous agitation. Alternatively, bound cells can be eluted by enzymatically “nicking” or digesting an enzyme-sensitive “spacer” sequence between the solid phase and the antibody. Spacers bound to agarose beads are commercially available from, for example, Pharmacia.

The eluted, enriched fraction of cells may then be washed with a buffer by centrifugation and either said enriched fraction or the unbound fraction may be cryopreserved in a viable state for later use according to conventional technology or introduced into the transplant recipient. The term ‘enriched’ is used to describe a population of cells in which the proportion of one particular cell type or the proportion of a number of particular cell types is increased when compared with the untreated population. The term ‘diminished’ is used to describe a population of cells in which the proportion of one particular cell type or the proportion of a number of particular cell types is decreased when compared with the untreated population.

The Composition of the Invention

The present invention further relates to a composition comprising a population containing cells originating from mammalian neural cells, wherein the percentage of FA1 expressing cells is at least 10%. Preferably, the percentage of FA1 expressing cells is at least 20%, more preferably at least 30%, more preferably at least 40%, more preferably at least 50%, more preferably at least 60%, more preferably at least 70%, more preferably at least 80% and most preferably at least 90%.

Furthermore, the present invention relates to a composition comprising a population containing cells originating from mammalian neural cells, wherein the percentage of dopaminergic cells is at least 20%. It is believed that the majority of FA1 expressing cells are dopaminergic cells. Preferably, the percentage of dopaminergic cells is at least 30%, more preferably at least 40%, more preferably at least 50%, more preferably at least 60%, more preferably at least 70%, more preferably at least 80% and most preferably at least 90%.

Also, the present invention relates to a composition comprising a population of cells obtainable by a method comprising the steps of:

a) combining a starting population containing cells originating from mammalian neural cells with antibodies which bind specifically to FA1 to produce a first cell mixture,

b) removing unbound antibodies from the first cell mixture to produce a second cell mixture, and

c) separating cells comprising FA1 antibodies from the second cell mixture to produce a cell population enriched in FA1 expressing cells and a cell population diminished in FA1 expressing cells.

In addition, the present invention relates to a FA1 expressing cell obtained by the method of the invention, wherein the FA1 expressing cell has been subjected to a genetic modification.

Uses of the Composition of the Invention

The present invention relates to the use of the composition of the invention or the cell of the invention for transplantation, for drug screening and for gene expression analysis. Furthermore, the present invention relates to the use of the composition of the invention or the cell of the invention as an immunogen for generation of antibodies.

Also, the present invention relates to an implantable encapsulated device comprising the composition of the invention or the cell of the invention.

Furthermore, the present invention relates to a method for measuring the content of FA1 expressing cells in a sample comprising the steps of:

a) combining a starting population containing cells originating from mammalian neural cells with antibodies which bind specifically to FA1 to produce a first cell mixture,

b) removing unbound antibodies from the first cell mixture to produce a second cell mixture,

c) separating cells comprising FA1 antibodies from the second cell mixture to produce a cell population enriched in FA1 expressing cells and a cell population diminished in FA1 expressing cells, and

d) determining the amount of FA1 expressing cells relative to the sum of the FA1 expressing cells and cells not expressing FA1. A preferred embodiment the said measuring method further comprises the procedure of measuring the content of cells expressing at least one lineage specific marker in the sample.

In addition, the present invention relates to a method of identifying FA1 expressing cells in a population containing cells originating from mammalian neural cells comprising contacting the cells with a labelled antibody to FA1/dlk1 and detecting the labelling.

As indicated above, one application for antibodies to FA1 is the isolation of a highly enriched source of dopaminergic neurons for transplantation into patients with Parkinson's disease.

The present invention contemplates the use of methods employing an FA1 antibody to separate dopaminergic neurons or neural stem/progenitor cells from other neural cells. Generally, a cell suspension prepared from human CNS tissue (e.g. from human fetal brain) is brought into contact with an FA1 antibody. Cells that have been bound by FA1 antibody are then separated from unbound cells by any means known to those skilled in the art. The CNS tissue may be taken from any part of the brain or spinal cord and may be selected by dissection of particular regions, which contain particular cell types. For instance the ventral mesencephalon may be selected to provide dopaminergic neurons and the substantia nigra pars compacta is particularly rich in dopaminergic neurons. The developing ventral mesencephalon may be particularly suitable for the enrichment of immature dopaminergic neurons and their commited progenitors. For the isolation of uncommitted neural stem and progenitor cells capable of differentiating into both glial and neuronal phenotypes, the periventricular regions of the developing brain, preferably the subventricular region of the forebrain, or germinal centers, such as the lateral ganglionic eminence, may be particular suitable as starting materials. In addition, the adult subventricular zone, the adult olfactory bulb and the hippocampus contain neural stem cells and progenitors capable of differentiating into both glial and neuronal phenotypes, which make these suitable anatomical tissue regions for FA1-based cell isolation. Glial cell may include both astrocytes and oligodendrocytes.

In a further embodiment, the invention provides cell populations useful in methods of ex vivo gene therapy. Expression vectors may be introduced into and expressed in these cells, or their genome may be modified by homologous or non-homologous recombination by methods known in the art. In this way, diseases may be treated, which are related to the lack of secreted proteins including, but not limited to hormones, enzymes, and growth factors. Inducible expression of a gene of interest under the control of an appropriate regulatory initiation region will allow production (and secretion) of the protein in a fashion similar to that in the cell that normally produces the protein in nature.

Antibodies that label the populations of neural stem cells, neural progenitor cells and their differentiated progeny are extremely useful in drug screening, gene discovery and for transplantation purposes because they allow the enrichment of populations of e.g. dopaminergic neurons or their progenitors in a single step. Cells recovered with FA1 antibody derived from different stages in their development could be used in studies on the mechanisms of action of cells, factors, and genes that regulate dopaminergic cell proliferation and differentiation. Furthermore, dopaminergic neurons from normal and pathological brain tissue may be recovered using FA1 antibodies and compared.

The above cell populations containing FA1 enriched cells can be used in therapeutic methods such as cell transplantation, as well as other methods that are readily apparent to those skilled in the art. Other uses envisaged for these cells are for drug screening, antibody production and gene discovery. The compositions of the invention may also be used to generate antibodies to the membrane bound portion of dlk1 which remains following proteolytic cleavage of dlk1 to give the soluble form. They may also be used in methods to identify the protease responsible for cleavage of dlk1. For example dlk1 could be expressed in a eukaryotic cell (e.g. yeast) that does not normally process it. The eukaryotic cell could then be contacted with fractionated cell extracts from FA1 producing cells, and the fraction which cleaves dlk1 could be identified and treated to isolate the said protease. The protease, which cleaves dlk1, could be a key element in the differentiation of primitive cell types. It is also envisaged that fractionated extracts containing the protease, obtained from enriched populations of FA1 producing cells of the invention, could be used to regulate the differentiation of stem- and progenitor cells.

In another embodiment, the FA1 antibody can be used to isolate FA1 enriched cells, which can be used in various protocols of genetic therapy.

Production of Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide resembling the immunogenic protein, or a recombinantly expressed immunogenic protein. Furthermore, the protein may be conjugated to a second protein that is known to be immunogenic in the mammal being immunized. Examples of such immunogenic proteins include but are not limited to keyhole limpet hemocyanin, serum albumin, bovine thyroglobulin, and soybean trypsin inhibitor.

The polyclonal antibodies directed against the immunogenic protein can be isolated from the mammal (e.g. from the blood) and further purified by well known techniques, such as affinity chromatography, using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography.

Monoclonal anti-FA1 cell antibodies can be produced readily by one skilled in the art. The general methodology for making monoclonal antibodies using hybridoma technology is now well known in the art. See, e.g., M. Schreier et al., Hybridoma Techniques (Cold Spring Harbor Laboratory 1980); Hammerling et al., Monoclonal Antibodies and T-Cell Hybridomas (Elsevier Biomedical Press 1981); Kennett et al., Monoclonal Antibodies (Plenum Press 1980). Immortal, antibody-secreting cell lines can also be produced by techniques other than fusion, such as direct transformation of B-lymphocytes with oncogenic DNA or EBV. Several antigen sources can be used, if desired, to challenge the normal B-lymphocyte population that is later converted to an immortal cell line.

The FA1 protein is expressed as a cell-surface antigen on many immature cell populations. FA1 may also be purified from amniotic fluid as a 32-38 kD glycoprotein. A purification method for mouse FA1 is given by Bachmann et al., (J Reprod and Fert 1996; 107:279-285) and this can also be used for human FA1.

For example, the purified FA1 from amniotic fluid may be used as an immunogen to challenge the mammal (e.g., mouse, rat, hamster, etc.) used as a source for normal B-lymphocytes. The antigen-stimulated B-lymphocytes are then harvested and fused to an immortal cell line or transformed into an immortal cell line by any appropriate technique. A preferred hybridoma producing the monoclonal FA1 antibody is produced by challenging a mouse with the FA1 antigen and fusing the recovered B-lymphocytes with an immortal myeloma cell such as X63Ag8.6.5.3 or SP2/0-Ag14. Antibody-producing immortal cells can be screened for appropriate antibody production by selecting clones that are strongly and specifically reactive with the dopaminergic neurons in the SN using sectioned human brain tissue and immunohistochemistry. Antibodies produced by clones, which show those properties can then be tested for reactivity towards other neural cell populations known to express FA1.

A mouse hybridoma producing monoclonal FA1 antibody (clone 142.2) is described in a previous publication (Jensen et al., Eur J. Biochem. 1994 Oct. 1; 225(1): 83-92.). Other Hybridomas producing FA1 antibodies are: F12, F15, F30, F59, F31, F32, F33, F38, F54, 142-1 (Jensen, et al. (submitted paper: J. Immun Methods, 2001) The immunogen to be used for the generation of antibodies against FA1/dlk1 maybe 1) intact, native FA1 as purified from any human physiological fluid (milk, amniotic fluid, serum, seminal plasma, follicular fluid, urine); 2) FA1 or smaller products purified from primary cell cultures or cell lines (including genetically engineered cells) that generate soluble dlk1 forms; 3) membrane fractions from cells that express all forms of dlk1; 4) synthetic peptides or fusion proteins encompassing parts of or the entire extracellular part of the multiple dlk1 forms or; 5) chimeric proteins presenting any dlk1 form as a dimer, which includes fusion proteins and hybridoma cell lines in which the secreted immunoglobulin molecule has been genetically modified so the Fab region has been replaced with dlk1 in any form.

Another alternative is to use an FA1 antibody in the production of monoclonal antibodies that recognize different antigens on dopaminergic cells of the SN or other FA1 expressing cell populations derived from the CNS. The cells isolated from the ventral midbrain or other brain regions with FA1 antibody can be used as an immunogen, as described above, to produce a panel of monoclonal antibodies against dopaminergic neurons. The production of such antibodies is greatly facilitated by the use of substantially pure populations of dopaminergic neurons provided by the FA1 antibody. The specificities of such antibodies can be determined readily through routine screening by one skilled in the art. Thus, additional surface markers for dopaminergic neurons and other FA1 expressing cell populations derived from the CNS (and antibodies to these antigens) can be identified by those skilled in the art.

The antibodies according to the subject invention may be either monoclonal, polyclonal, or a mixture of monoclonal and/or polyclonal antibodies. The antibody may comprise whole antibody or antigen-binding fragments thereof, such as Fab.sub.2, Fab and Fv fragments. Antigen binding fragments can be prepared using conventional techniques known in the art, such as proteolytic digestion of antibody by papain or pepsin, or through standard genetic engineering techniques known in the art. Monoclonal antibodies exemplified herein can be engineered so as to change the isotype of the antibody. For example, an IgG.sub.2A isotype can be engineered as an IgG.sub.1, IgG.sub.2B, or other isotypes. Also contemplated by the subject invention are antibodies that are reactive with the FA1 antibody and which have been engineered to comprise human antibody constant regions. “Humanized” antibodies can be prepared using standard methods known in the art. See, for example, U.S. Pat. No. 5,585,089 (issued Dec. 17, 1996), the disclosure of which is hereby incorporated by reference.

### EXAMPLES

The following examples are provided to illustrate specific embodiments of the present invention. The examples are included for illustrative purposes only, and are not intended to limit the scope of the present invention.

### Example 1

Immunocytochemistry Using Human Cell Cultures Containing Neural Stem- and Progenitor Cells

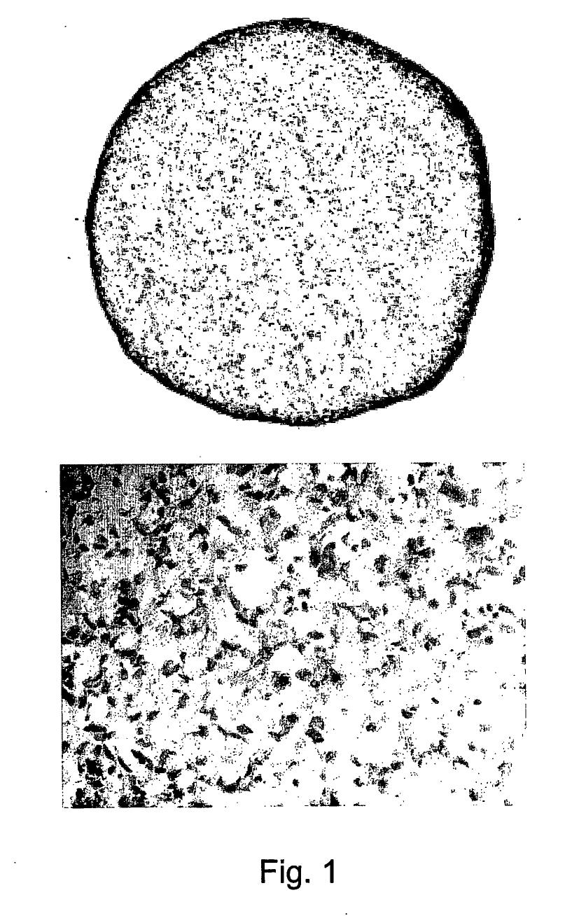

Pellets of neural stem/progenitor cell cultures established from human fetal forebrain tissue expanded as free-floating aggregates were collected by centrifugation, formalin-fixed and embedded in tissue tech, cut in 14 μm sections and mounted on glass slides. Endogenous peroxidase activity was blocked with H₂O₂/methanol. Sections were incubated with a polyclonal rabbit anti-FA1 antibody diluted 1:100 and subsequently reacted with a biotinylated secondary antibody (swine anti-rabbit IgG (DAKO E0353, diluted 1:400). The sections were then incubated with an avidin-biotin conjugated horseradish peroxidase complex (Vectastain ABC kit, Vector Laboratories) and developed using DAB as chromogen. Cells were permeabilized by including 0.3% Triton in the incubation buffer. Many cells located at the edges and isolated cells within the spheres were staining strongly positive for FA1 (FIG. 1A). In the intensely stained cells, the immunoreactivity was seen in several subcellular localizations but absent in the nucleus (FIG. 1B).

In another embodiment, the adherent, nestin-positive, human neural stem cell line, HNSC.100 (Villa et al., 2000), was plated onto PLL coated coverslips in proliferation medium and fixed in 4% PFA, washed three times with PBS, permeabilized in 100% ethanol, washed three times with PBS and incubated with polyclonal rabbit anti-FA1 antibody diluted 1:200 in PBS with 2% BSA and 0.3% Triton-X-100. After 3 washes with PBS, cells were incubated for 30 minutes at room temperature with cyanin-3-conjugated goat-anti-rabbit IgG (1:500; Chemicon).

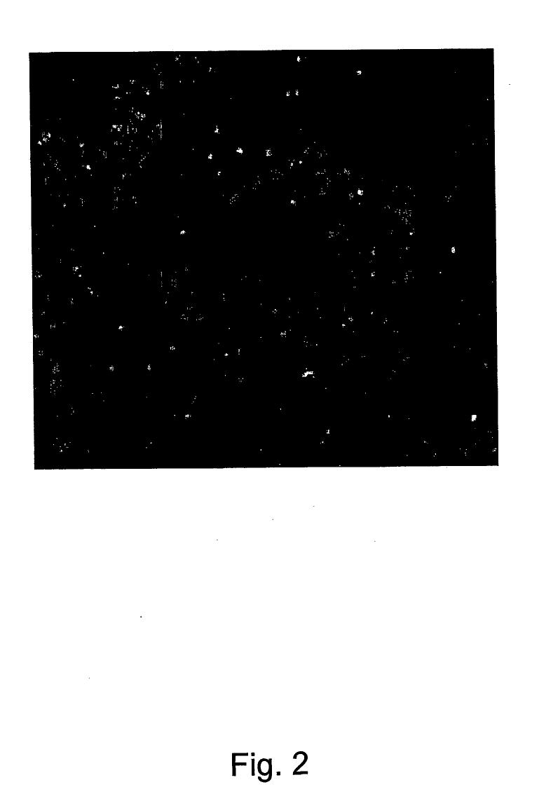

Coverslips were washed three times with PBS and counterstained with Hoechst nuclear dye. FA1 immunoreactivity was observed in 0.5-1% of the HNSC.100 population with the FA1 staining mainly located to the peri-nuclear area (FIG. 2).

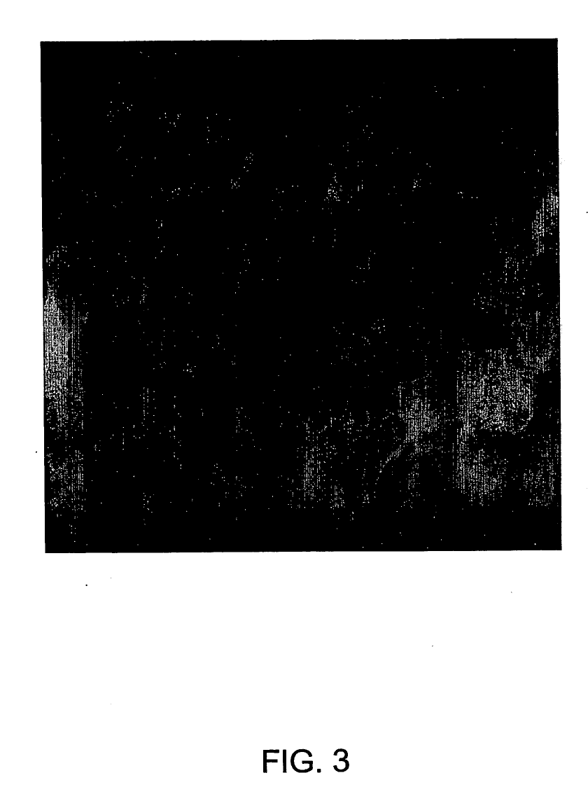

A third embodiment includes adherent cultures with glial characteristics derived from the embryonic human lateral ganglionic eminence (LGE) plated onto coverslips in proliferation medium. Surface-labelling of these cultures with FA1 antibodies were done by washing with PBS, first at room temperature and then at 4° C. and surface-labeled for 30 min at 4° C. by the addition of polyclonal rabbit anti-FA1 antibody diluted 1:100 in ice cold PBS. Cells were washed 3 times with PBS and fixed in ice cold 4% PFA, where after the coverslips were placed at room temperature for 30 minutes. The fixative was removed by 3 washes with PBS. Cells were then incubated for 30 minutes at room temperature with Cyanin-3 conjugated goat-anti-rabbit IgG (1:500; Chemicon). Coverslips were washed three times with PBS, and counterstained with Hoechst nuclear dye. Cells displaying distinct membrane localization of the FA1 immunoreactivity were detected in 0.5-1% of the LGE cultures (FIG. 3).

### Example 2

Immunocytochemistry Using TH Induced Cultures Derived from Cultures of Human Neural Stem- and Progenitor Cells

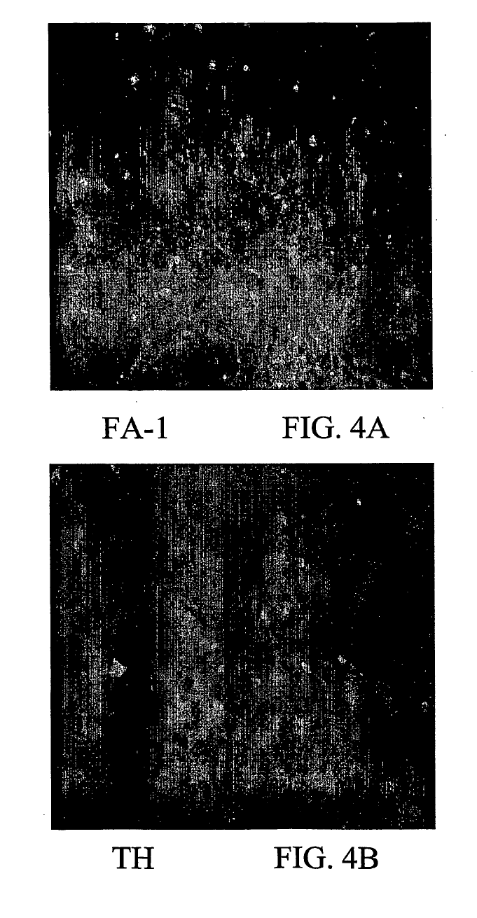

Human neural stem- and progenitor cultures grown as free-floating aggregates were plated and treated as described in PCT/DK02/00262 (Meijer et al.). This allows induction of 5-10% TH positive neurons. In parallel, cultures were differentiated using the standard protocol (plating on PLL/laminin in medium without TH inducing factors). After three days, cells were fixed in 4% PFA (paraformaldehyde), washed with PBS, permeabilized with 100% ethanol, washed three times with PBS, and resuspended in PBS with 5% BSA and 0.3% triton-X-100 for 60 minutes at 4° C. Then the cells were incubated with polyclonal rabbit anti-FA1 antibody diluted 1:100 in PBS with 2% BSA and 0.3% Triton-X-100. Unbound antibody was removed by 3 washes with PBS. Cells were then incubated for 30 min at 4° C. with Cyanin-3 conjugated goat-anti-rabbit IgG (1:500; Chemicon) and nuclei counterstained using Hoechst nuclear dye. FA1 staining of TH induced cultures showed that 10-15% of the total cell population displayed strong FA1 immunoreactivity. FA1-positive cells were mainly found within the population of cells that had migrated out from the plated spheres and differentiated (FIG. 4A). Strong FA1 staining was seen in the peri-nuclear area but also localization to the processes was observed. In contrast only a few FA1 positive cells could be observed in control cultures differentiated under conditions that do not allow induction of TH and only in the edges of the plated spheres (FIG. 4B).

### Example 3

Surface Labelling of TH Induced Human Neural Progenitors

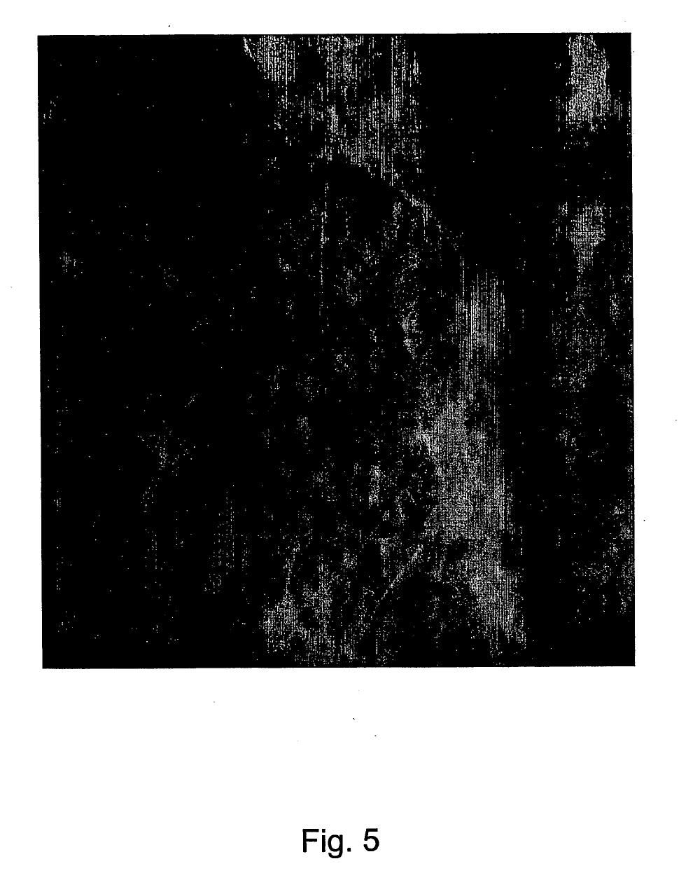

To label the surface of neurospheres, cultures TH-induced on glass coverslips as described above were washed with PBS, first at room temperature and then at 4° C., and surface-labeled for 30 min at 4° C. by the addition of the polyclonal rabbit anti-FA1 antibody diluted 1:100 in ice cold PBS. Cells were washed 3 times with PBS and fixed in ice cold 4% PFA, where after the coverslips were placed at room temperature for 30 minutes. The fixative was removed by 3 washes in PBS. Cells were then incubated for 30 minutes at room temperature with Cyanin-3 conjugated goat-anti-rabbit IgG (1:500; Chemicon) diluted in PBS. Coverslips were washed three times with PBS and counterstained with Hoechst nuclear dye. FA1 immunostaining could be localized to the cell surface of a small section of neurospheres grown under conditions that induce the expression of TH (FIG. 5), whereas cell surface labeling with FA1 antibody was not observed in control cultures that had not been exposed to TH inducing conditions. The cell surface staining for the FA1 antigen was localized to dendritic formations in addition to cell bodies of differentiated neurospheres.

### Example 4

Immunohistochemistry on Sections of Mouse, Rat and Human Brain Tissue

Samples of normal human brain stem tissue obtained according to approval by the regional science ethical committee for Vejle and Funen counties, was long-term fixed in phosphate-buffered formalin and embedded in paraffin for immuno-histochemical analysis. Rat brain tissue was perfusion fixed in 4% paraformaldehyde (PFA), cryoprotected by immersion in 25% sucrose and cut in 40 μm coronal sections. Immunohistochemistry was carried out on free-floating sections. Endogenous peroxidase activity was blocked with 3% H₂O₂/10% methanol followed by pre incubation for 1 hour in KPBS with 2% normal goat serum and 0.25% Triton X-100. Sections were the incubated with a primary monospecific rabbit anti-rat FA1 antibody (1:3000) and subsequently reacted with a biotinylated secondary antibody (swine anti-rabbit IgG (DAKO E0353, diluted 1:500). The sections were then incubated with an avidin-biotin-peroxidase (ABC-kit Vector laboratories, CA, USA), and finally visualized using 3′3-diaminobenzidine (Sigma, MO, USA) as chromogen. For co-localization of FA1/dlk1 and tyrosine hydroxylase, sections were incubated with primary rabbit anti-rat FA1 (1:3000) and mouse-anti-TH (1:2000; Chemicon, USA), followed by incubation with FITC conjugated donkey-anti-rabbit (1:400; Jackson Immunoresearch, USA) and Cy3 conjugated sheep-anti-mouse (1:400; Jackson Immunoresearch, USA).

Following staining all sections were mounted onto glass slides for microscopic evaluation. Embryonic mouse brain was fixed in 4% paraformaldehyde (PFA), cryoprotected by immersion in 30% sucrose and cut in 12 μm coronal sections. Immunohistochemistry was carried out on serial mounted sections. Endogenous peroxidase activity was blocked with 3% H₂O₂/10% methanol followed by pre incubation for 1 hour in KPBS with 2% normal goat serum and 0.25% Triton X-100. Sections were the incubated with either of the primary antibodies rabbit anti-rat FA1 (1:3000) or rabbit-anti-TH (1:1000; Pelfreeze, USA) and subsequently reacted with a biotinylated secondary antibody (swine anti-rabbit IgG (DAKO E0353, diluted 1:500). The sections were then incubated with an avidin-biotin-peroxidase (ABC-kit, Vector laboratories, CA, USA), and finally visualized using 3′3-diaminobenzidine (Sigma, MO, USA) as chromogen.

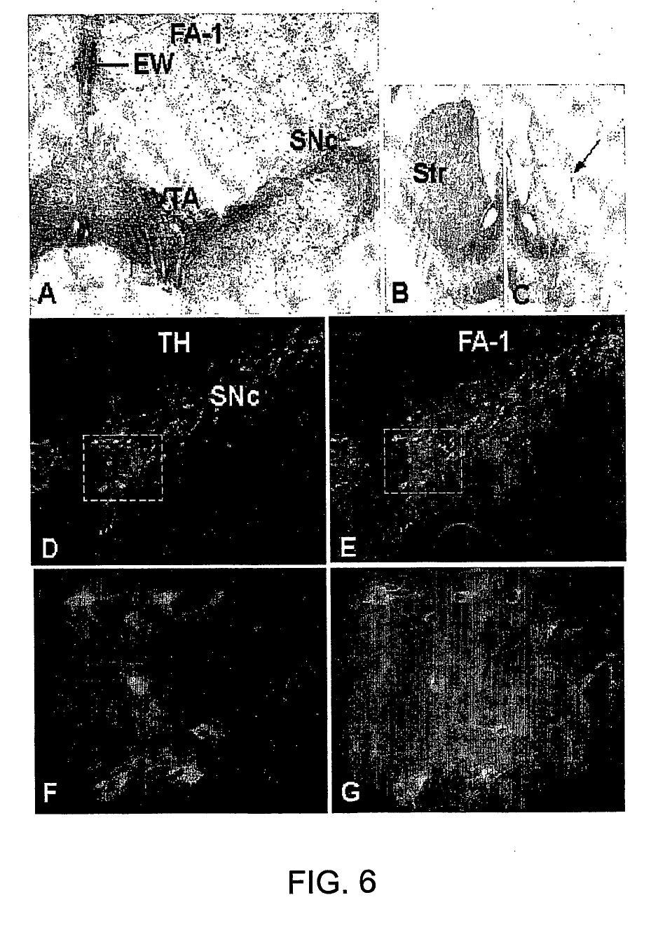

The localization of FA1/dlk1 was analyzed by immunohistochemistry (IHC) on formalin fixed and paraffin sections of the mouse, rat and human brain stem. In all species strong immunoreactivity to FA1 was observed in selected groups of neurons only. In sections of the rat mesencephalon FA1-positive neurons were mainly confined to the Edinger-Westphal nuclei (EW), the ventral tegmental area (VTA) and pars compacta of substantia nigra (SNc) (FIG. 6). Only few neurons in the reticular part of the substantia nigra (SNr) were FA1-positive (FIG. 4A).

The same localization of FA1-positive neurons was found in sections of the human mesencephalon. In both species, FA1 immunoreactivity was seen in the cell body and the dendritical/axonal processes of positive neurons. Double labeling with FA1 and TH showed a high degree of co-localization (FIG. 6D-G). In the rat brain FA1 positive terminals in the striatum (FIG. 6B-C terminal area for nigral dopaminergic neurons) were absent around the site of injection (arrow in FIG. 6C) of the dopamine-specific toxin 6-hydroxydopamine.

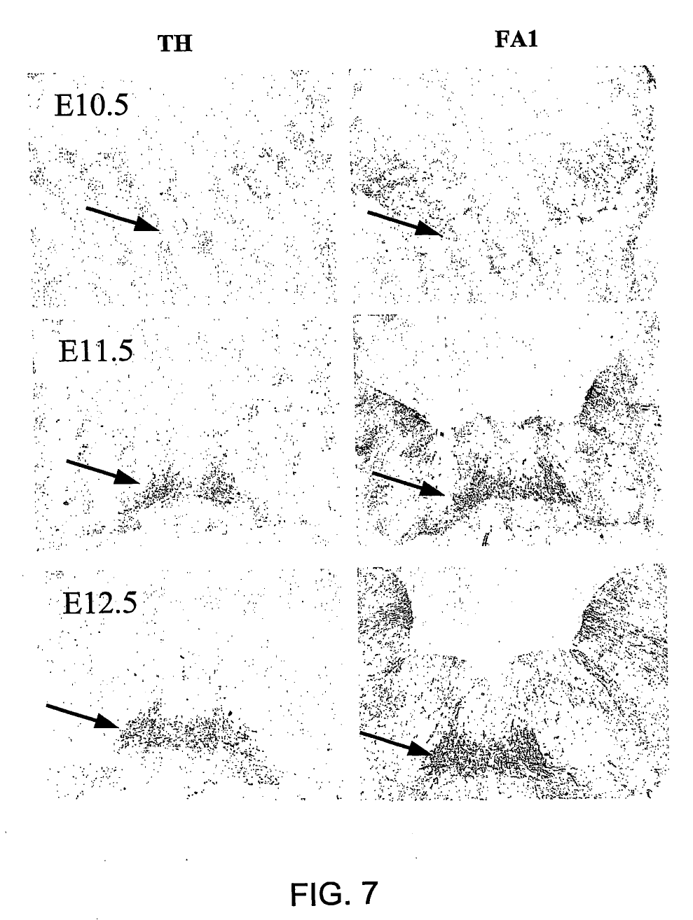

FIG. 7 depicts cross-sections through the embryonic mouse midbrain at three different stages of development, E10.5, 11.5 and 12.5. The pictures to the left show tyrosine hydroxylase (1 labelling and the pictures to the right show FA1 labeling. The ventral mesencephalic area is marked by arrows.

In sections of the mouse embryonic mesencephalon, FA-1-labeling was seen to co-localize with and follow the developmental pattern of the dopaminergic cells identified with TH-labeling (FIG. 7).

### Example 5

Preparation of Primary Cell Culture for Cell Sorting

Dissected nervous tissue was transferred to a Petri dish with D-PBS (Gibco cat # 14190-094) and cut into 1 mm³ pieces. The pieces of tissue was transferred to a 15 ml tube containing 3 ml of D-PBS and 20 μg/ml DNase (Sigma cat # D-4513) and pipetted up and down 10-12 times with a Pasteur pipette to dissociate the pieces into a cell suspension. The cell suspension was filtered through a 70 μm nylon filter (Falcon cat # 2350) to obtain a single cell suspension.

### Example 6

Sorting of FA1-Expressing Cells in Proliferating Neurospheres by FACS Analysis

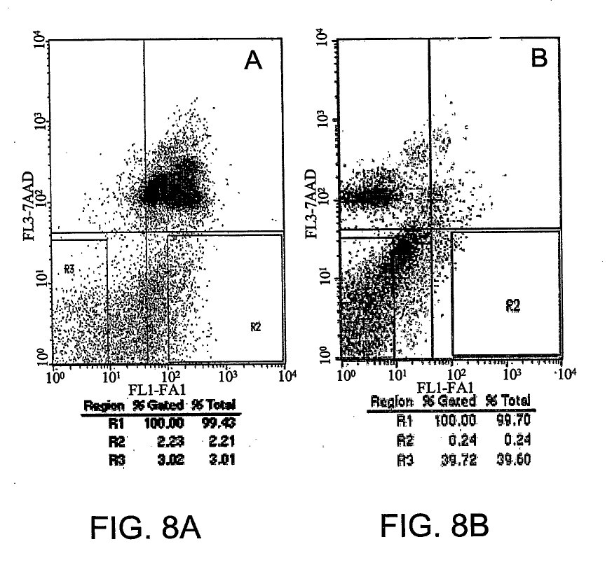

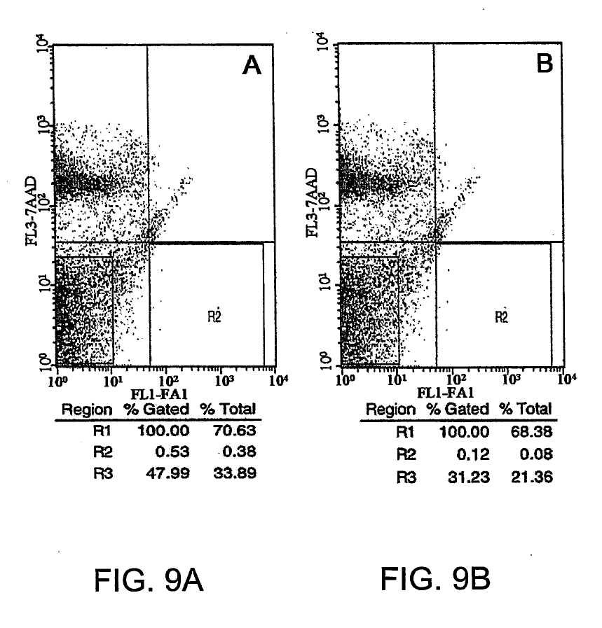

Human neural stem and progenitor cells grown as free-floating aggregates were dissociated to a single cell suspension using mechanical dissociation Small clumps of cells were removed by filtering through a 30 μm nylon mesh. Cells were resuspended in PBS with 5% BSA and 5 mM EDTA for 60 minutes at 4° C., after which the cells were incubated with polyclonal rabbit anti-FA1 antibody diluted 1:20 in PBS with 2% BSA and 5 mM EDTA for 120 min on ice. Cells were washed three times with washing buffer (PBS with 2% BSA and 5 mM EDTA) and incubated with FITC conjugated goat anti-rabbit antibody (Jackson) diluted in washing buffer for 30 min on ice in the dark. After washing, the cells were resuspended in washing buffer and sorted by flow cytometry using FACSVantage (Becton Dickinson). FACS analysis showed that 0.5-2.2% of the proliferating neurospheres expressed membrane-bound FA1 (R2 in FIG. 8A and FIG. 9A), whereas only 0.1-0.2% of the negative control cells were detected in that same region (R2 in FIG. 8B and FIG. 9B).

### Example 7

Sorting of FA1 Expressing Cells in TH-Induced Neurosphere Population by FACS Analysis

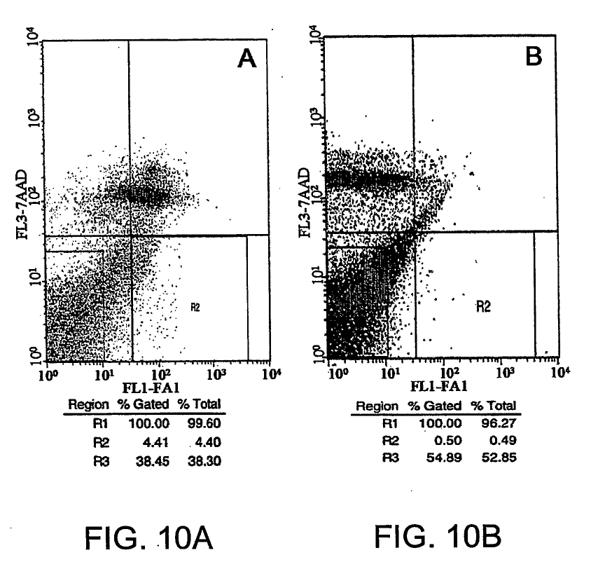

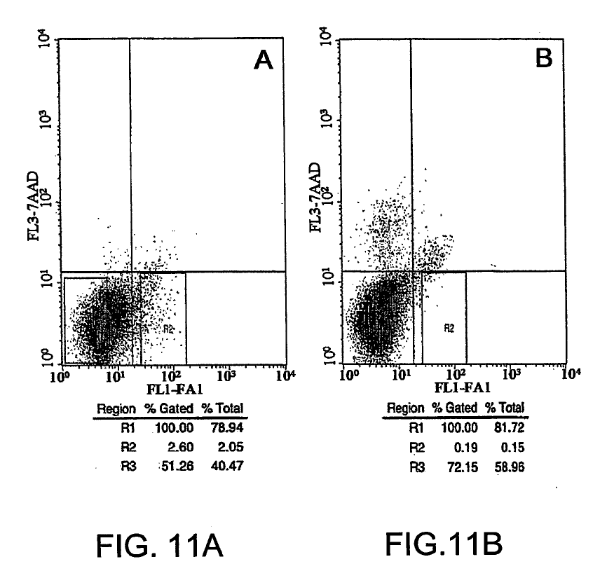

Human neural stem- and progenitor cultures grown as free-floating aggregates were plated onto coated surfaces in TH-induction medium as described in Example 2. The cells were harvested by Trypsin/EDTA (Sigma) and small clumps of cells were removed by filtering through a 30 μm nylon mesh. Cells were resuspended in PBS with 5% BSA and 5 mM EDTA (blocking buffer) for 60 minutes on ice, centrifuged and incubated with polyclonal rabbit anti-FA1 diluted 1:20 in PBS with 2% BSA and 5 mM EDTA (washing buffer) for 120 min on ice. Cells were washed three times with cold wash buffer and incubated with FITC conjugated goat anti-rabbit antibody (Jackson) diluted in washing buffer for 30 min on ice. After washing, differentiated FA1-positive cells were selected using the FACSVantage (Becton Dickinson) (FIG. 10). The FACS analysis showed that 4.4% of the TH-induced cells expressed membrane-bound FA1; these FA1-positive cells were isolated and reanalyzed in the FACSVantage (FIG. 11), showing an enrichment of FA1-positive cells (73%).

### Example 8

Sorting of FA1 Expressing Cells in HNSC.100 Population by FACS Analysis

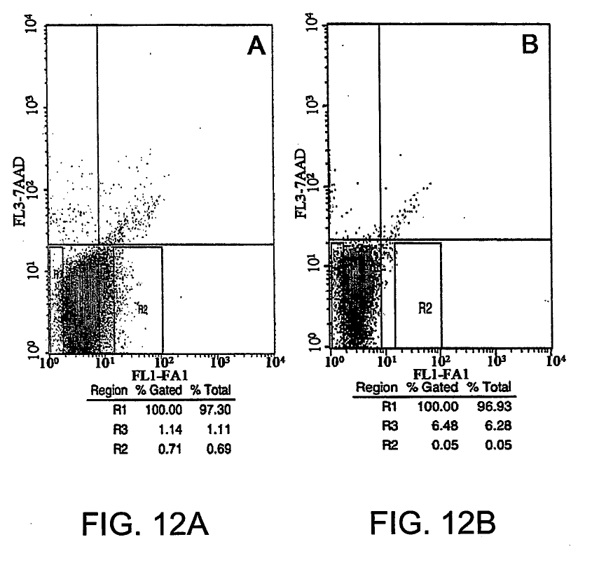

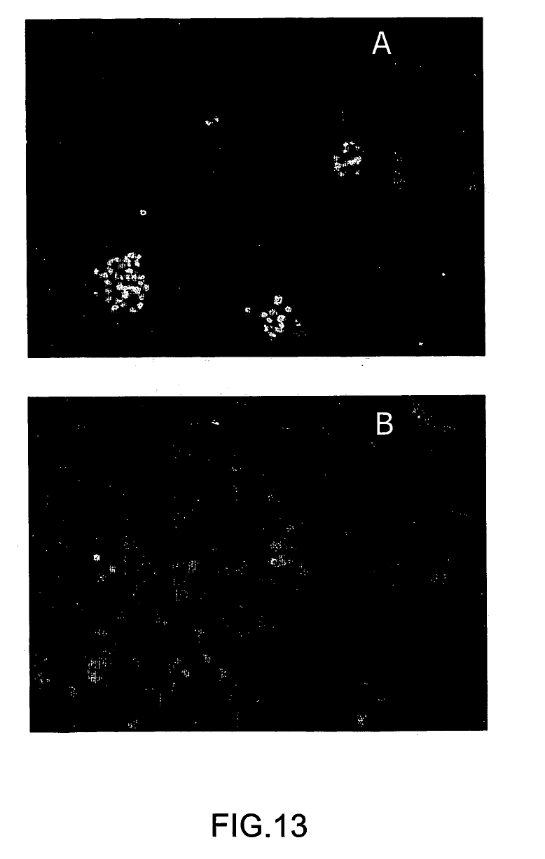

Proliferating cultures of the human neural stem cell line, HNSC.100 (Villa et al., 2000), were harvested from flasks using trypsin/EDTA (Sigma). Small clumps of cells were removed by filtering through a 30 μm nylon mesh. The cells were resuspended in PBS with 5% BSA and 5 mM EDTA (blocking buffer) for 60 min on ice, centrifuged and incubated with polyclonal rabbit anti-FA1 antibody diluted 1:20 in PBS with 2% BSA and 5 mM EDTA (washing buffer). Cells were washed three times with cold washing buffer and incubated with FITC conjugated goat anti-rabbit antibody (Jackson) for 30 min on ice. After washing three times with washing buffer, the cells were sorted by flow cytometry using FACSVantage (Becton Dickinson) (FIGS. 12 and 13). FACS analysis showed that 0.7-2.6% of the HNSC.100 cell line could be isolated based upon expression of membrane-bound FA1 (R2), where as 0.1-0.2% of the negative control cells were located in R2.

HNSC.100 cells sorted for FA1 immunoreactivity were resuspended in standard proliferation medium and plated onto PLL coated coverslips. After 3 days of incubation, cells were fixed and processed for immunocytochemistry using polyclonal rabbit anti-FA1 antibody (FIG. 14). An enrichment of FA1-positive cells was observed in the population of cells sorted for FA1 immunoreactivity with a clustering staining pattern (FIG. 14A), indicating cell doublings after cell sorting.

1. A method of obtaining a cell population enriched or diminished in FA1 (dlk1) expressing cells comprising the steps of:
a) combining a starting population containing cells originating from mammalian neural cells with antibodies which bind specifically to FA1 to produce a first cell mixture,
b) removing unbound antibodies from the first cell mixture to produce a second cell mixture, and
c) separating cells comprising FA1 antibodies from the second cell mixture to produce a cell population enriched in FA1 expressing cells and a cell population diminished in FA1 expressing cells.

2. A method according to claim 1, wherein the starting cell population is selected from the group consisting of:
(i) a population of primary neural cells,
(ii) a population of in vitro cultured neural stem- and/or progenitor cells, and
(iii) a population of in vitro differentiated neural stem and/or progenitor cells.

3. A method according to claim 1, wherein the cells of the starting population originate from human tissue.

4. A method according to claim 1, wherein the cells of the starting population are fetal cells.

5. A method according to claim 1, wherein the cells of the starting population are CNS cells.

6. A method according to claim 1, wherein the FA1 specific antibodies are monoclonal.

7. A method according to claim 1, wherein the antibodies are labelled.

8. A method according to claim 7, wherein the antibodies are coupled to a fluorescent compound.

9. A method according to claim 7, wherein the antibodies are biotinylated.

10. A method according to claim 7, wherein the antibodies are linked to a solid particle.

11. A method according to claim 10, wherein the solid particle is a magnetic particle.

12. A method according to claim 7, wherein the separation of cells comprising FA1 antibodies is carried out by a mechanical cell sorter.

13. A method according to claim 8, wherein the separation of cells comprising FA1 antibodies is carried out using a fluorescence-activated cell sorter (FACS).

14. A method according to claim 9, wherein prior to step c) the second cell mixture is contacted with a streptavidin-fluorochrome, and wherein the separation of cells comprising FA1 antibodies is carried out using a fluorescence-activated cell sorter (FACS).

15. A method according to claim 9, wherein prior to step c) the second cell mixture is contacted with streptavidin linked to a particle, and wherein the separation of cells comprising FA1 antibodies is carried out by separating the particulate phase from the liquid phase.

16. A method according to claim 10, wherein the separation of cells comprising FA1 antibodies is carried out by separating the particulate phase from the liquid phase.

17. A method according to claim 1, wherein prior to step c) the second cell mixture is contacted with an antibody to the FA1 specific antibody linked to a particle, and wherein the separation of cells comprising FA1 antibodies is carried out by separating the particulate phase from the liquid phase.

18. A method according to claim 17, wherein the particle is a magnetic particle.

19. A method according to claim 1, wherein the cells of the starting population are adherent cells cultivated on a solid support, and wherein the removal of unbound antibodies is carried out by rinsing.

20. A method according to claim 1, wherein the cells of the starting population are cultivated in suspension, and wherein the removal of unbound antibodies is carried out by centrifugating the first cell mixture and separating off the resulting supernatant.

21. A method according to claim 1, wherein the starting cell population is subjected to a further cell sorting procedure to enrich or diminish the cell population in cells expressing at least one further lineage specific marker.

22. A method according to claim 21, wherein the further lineage specific marker is CD133.

23. A method according to claim 1, wherein the starting cell population originates from the ventral mesencephalon.

24. A method according to claims 1, wherein the starting cell population originates from the periventricular regions.

25. A composition comprising a population containing cells originating from mammalian neural cells, wherein the percentage of FA1 expressing cells is at least 10%.

26. A composition comprising a population containing cells originating from mammalian neural cells, wherein the percentage of dopaminergic cells is at least 20%.

27. A composition comprising a population of cells obtainable by a method comprising the steps of:
a) combining a starting population containing cells originating from mammalian neural cells with antibodies which bind specifically to FA1 to produce a first cell mixture,
b) removing unbound antibodies from the first cell mixture to produce a second cell mixture, and
c) separating cells comprising FA1 antibodies from the second cell mixture to produce a cell population enriched in FA1 expressing cells and a cell population diminished in FA1 expressing cells.

28. An FA1 expressing cell obtained by the method of claim 1, wherein the FA1 expressing cell has been subjected to a genetic modification.

29-32. (Canceled)

33. An implantable encapsulated device comprising the composition according to claim 25.

34-36. (Canceled)

37. An implantable encapsulated device comprising the composition according to claim 26.
